(19) United States
(12) Patent Application Publication
(10) Pub. No.: US 2011/0064782 A1
(43) Pub. Date: Mar. 17, 2011

(54) METHODS AND COMPOSITIONS FOR TISSUE REGENERATION

(76) Inventors: Stephen Bloor, Alwoodley (GB); Joanne Louise Proffitt, Alwoodley (GB); Paul Armitage, Ackworth (GB); Christine Elizabeth Dawson, Byram (GB)

(21) Appl. No.: 12/596,182

(22) PCT Filed: Apr. 15, 2008
(86) PCT No.: PCT/GB08/01315

§ 371 Date: Nov. 30, 2010

(30) Foreign Application Priority Data
Apr. 16, 2007 (GB) 0707234.1
Apr. 16, 2007 (GB) 0707235.8
Oct. 31, 2007 (GB) 0721347.3

(51) Int. Cl. A61K 9/00; C07K 14/78; C12P 21/06; A61K 38/39; C07K 1/14; A61P 19/00; A61P 43/00

(52) U.S. Cl. 424/423; 530/356; 435/68.1; 514/17.2

(57) ABSTRACT

A decellularised collagen-containing matrix for guided tissue regeneration, wherein the matrix is derived from a natural tissue material and is substantially free of non-fibrous tissue proteins, cellular elements and lipids or lipid residues and wherein the matrix displays the original collagen fibre architecture and molecular ultrastructure of the natural tissue material from which it is derived. The decellularised collagen-containing matrix is useful as an implant for guided tissue regeneration, having a capacity to induce guided regeneration of host tissue.

The present invention relates to tissue regeneration.

Implantable materials are used in a range of surgical applications, including replacement, reconstruction or repair of different body tissues. It is desirable that body tissue at an implant site be regenerated in an ordered manner to achieve good integration of the implanted material and effective replacement, reconstruction or repair of the body tissue.

According to a first aspect of the present invention there is provided a decellularised collagen-containing matrix for guided tissue regeneration, wherein the matrix is derived from a natural tissue material and is substantially free of non-fibrous tissue proteins, cellular elements and lipids or lipid residues and wherein the matrix displays the original collagen fibre architecture and molecular ultrastructure of the natural tissue material from which it is derived.

The decellularised matrix may optionally contain a portion of elastin. The proportion of elastin relative to collagen varies depending upon the nature and composition of the starting material. By way of example, ligaments and tendons may comprise as much as 90% collagen, dermis around 80% collagen, carotid artery around 50% collagen, and bone around 30% collagen. Typically, collagen is a major component of the processed tissues.

The decellularised collagen-containing matrix is useful as an implant for guided tissue regeneration, having a capacity to induce guided regeneration of host tissue.

According to a second aspect of the present invention there is provided an implant comprising a decellularised collagen-containing matrix, wherein the matrix is derived from a natural tissue material and is substantially free of non-fibrous tissue proteins, cellular elements and lipids or lipid residues and wherein the matrix displays the original collagen fibre architecture and molecular ultrastructure of the natural tissue material from which it is derived, characterised in that the matrix has a capacity to induce guided tissue regeneration.

According to a further aspect of the present invention there is provided a process for the manufacture of a decellularised collagen-containing matrix for guided tissue regeneration, which comprises treating a fibrous collagen-containing tissue material to remove therefrom cells and cellular elements, non-fibrous tissue proteins, lipids and lipid residues.

Whilst any appropriate processing methodology may be used, a particularly suitable process which may be adapted for use in preparing the decellularised collagen matrix for guided tissue regeneration is disclosed in U.S. Pat. No. 5,397,353, the contents of which are incorporated herein by reference. U.S. Pat. No. 5,397,353 describes processing of porcine dermal tissue to provide collagenous implant materials suitable for homo- or hetero-transplantation. The implants retain the natural structure and original architecture of the natural collagenous tissue from which they are derived, so that the molecular ultrastructure of the collagen is retained. The implant materials are long-lived and non-reactive, any reactive pathological factors having been removed, and provide an essentially inert scaffold into which host cells infiltrate readily following implantation.

It has now been found that the processing techniques of U.S. Pat. No. 5,397,353 may be used to provide a collagen-containing matrix which is capable of inducing guided tissue regeneration following implantation into a host. When a decellularised collagen-containing matrix according to the present invention is implanted into a host, it is rapidly infiltrated by host cells. It has surprisingly been observed that host cells within the implanted collagen-containing matrix have cellular characteristics of the natural tissue material from which the matrix is derived which may in some circumstances be different from the characteristics typical of the surrounding tissue at the site of implantation. Thus, following implantation, the growth and development of host tissue in and on the collagen-containing matrix is at least initially ‘guided’ by the implanted matrix. This is particularly surprising in view of the fact that the collagen-containing matrix is treated to remove non-fibrous tissue proteins, such as growth factors. As such, it would be expected that any molecular signals which could drive tissue-specific regeneration would be stripped from the collagen-containing matrix during processing and that exogenous factors such as growth factors would need to be added to the matrix in order to introduce the capacity to drive guided tissue regeneration. However, it would seem that some signalling functionality remains despite the tissue processing. Advantageously, the capacity of the collagen-containing matrix as described herein to induce guided tissue regeneration does not rely upon the addition of exogenous growth factors. Thus, in some embodiments the collagen-containing matrix may be free from exogenous growth factors.

The guided tissue regeneration means that the behaviour of cells and tissues in and on the implanted matrix is influenced by the matrix. The matrix exerts a tissue-specific influence, to guide the development of the regenerated tissue, providing for natural, ordered regeneration.

Without wishing to be bound by any particular theory, it seems possible that the host cells may be responding to ‘signals’ provided by the structure of the matrix itself, such that behaviour of host cells may be influenced, and tissue growth guided, by tissue-specific elements of the matrix structure, in particular the collagen and any elastin. It is hypothesised that such ‘signals’ may play a role in differentiation of host cells, including but not limited to progenitor cells, stem cells and differentiated cells of the local environment. The signals may be recognised directly by host cells. It is also possible that elements of the matrix structure act indirectly on the host cells, perhaps by binding growth factors or signalling molecules in a tissue-specific manner. The signals may reside in a combination of one or more primary, secondary, tertiary or quaternary structural elements of the fibrous tissue proteins of the matrix. As such, signalling may be occurring through recognition of a combination of one or more of: protein sequences, one-dimensional topography, two-dimensional topography or three-dimensional topography.

Following implantation of the matrix into a host, the site of implantation is a complex and continually changing environment. It has been observed that the host cells within the implanted collagen-containing matrix have cellular characteristics of the natural tissue material from which the matrix is derived. Where the matrix is implanted into tissue of a different type from the natural tissue material from which the matrix is derived, it is likely that the initial influence of the matrix on growth and development of the regenerating host tissue will eventually be overtaken by signals from the surrounding tissue environment. In such circumstances, even though the initial development of the host tissue may show characteristics of the tissue from which the matrix is derived rather than the tissue at the site of implantation, it is likely that the host tissue will take on the appropriate characteristics of the surrounding tissue as the regeneration processes ensue.

Of course, where the collagen-containing matrix is implanted into a site of the same or a similar tissue as the natural tissue from which the matrix is derived, the initial tissue regeneration will be appropriate to the site of implantation, and subsequent growth and regeneration may follow generally the pathways already initiated, the environment and cell signals being correct for regeneration of the tissue in question.

The collagen-containing matrix as herein described may also usefully be employed for in vitro regeneration of tissues.

The present invention may be used to provide a collagen-containing matrix derived from any tissue. The tissue may be a non-dermal tissue. Dermis is a relatively simple structure, in which there is essentially a single layer of interwoven fibres of collagen and some elastin fibres. Advantageously, the present invention may provide a collagen-containing matrix derived from more complex tissues with more than one different collagen-containing (and optionally elastin-containing) components or sub-components.

By way of example only, suitable starting materials may include vascular tissue, bone, ligaments and tendons (which are effectively interchangeable in the context of the present invention), nerves, and bowel tissue. The invention may equally be used in relation to whole organs or parts of organs, and the term “tissue material” therefore encompasses organs or parts thereof. A decellularised collagen-containing matrix may be provided which retains the general three-dimensional structure of an organ, or part thereof, the structural material being essentially collagen with varying proportions of elastin and other fibrous tissue proteins. The organ may be any organ, or part thereof. Non-limiting examples include heart, liver, kidney, pancreas, spleen and bladder, and any vessel or tubular body structure, including blood vessels, gastrointestinal tract and urinary tubes, in particular the urethra and ureter.

The starting materials may be obtained from any human or non-human mammal. In some embodiments, it is preferred that porcine tissue materials are processed to provide the collagen-containing matrix compositions, although it will be understood that other mammalian sources may alternatively be employed, such as primates, cows, sheep, horses and goats.

Non-fibrous tissue proteins include glycoproteins, proteoglycans, globular proteins and the like. Cellular elements can include antigenic proteins and enzymes and other cellular debris arising from the processing conditions. These portions of the natural tissue material may be removed by treatment with a proteolytic enzyme.

Whilst any proteolytic enzyme which under the conditions of the process will remove non-fibrous tissue proteins can be used, the preferred proteolytic enzyme is trypsin. It has previously been found that above 20° C. the treatment can in some circumstances result in an alteration of the collagen fibre structure leading to a lower physical strength. Moreover, low temperatures discourage the growth of microorganisms in the preparation. It is therefore preferred to carry out the treatment with trypsin at a temperature below 20° C. Moreover, trypsin is more stable below 20° C. and lower amounts of it may be required. Any suitable trypsin concentration may be used, for instance a concentration within the range of around 0.01 g/L to 25 g/L. It has been found that good results can be obtained using 2.5 g/L porcine trypsin, pH 8.

In the context of dermal tissue processing, U.S. Pat. No. 5,397,353 teaches that the tissue should be digested with trypsin over a period of 28 days. However, this has been found to be unsuitable for treatment of certain tissues, as over-exposure to trypsin can damage the overall integrity of the implant. As such, it may be necessary to reduce the digestion time for certain tissue types, notably blood vessels. It is generally necessary to digest the tissue with trypsin for at least one hour.

It will be appreciated that the reaction conditions for the treatment with trypsin may be routinely adjusted.

One method of removing lipids and lipid residues from the collagenous tissue is by the use of a selective enzyme such as lipase. A further, simpler and preferred method is solvent extraction using an organic solvent. Non-limiting examples of suitable solvents include non-aqueous solvents such as acetone, ethanol, ether, or mixtures thereof.

The method may be used to process collagen-containing tissue material to provide a decellularised collagen-containing matrix that is substantially free of non-fibrous tissue proteins, cellular elements, and lipids or lipid residues. Those substances said to be “substantially free” of materials generally contain less than 10% of, more typically less than 5% of, and preferably less than 1% of said materials.

The tissue processing may optionally include a step of treatment with a cross-linking agent. Whilst any cross-linking agent may be used, preferred cross-linking agents include polyisocyanates, in particular diisocyanates which include aliphatic, aromatic and alicyclic diisocyanates as exemplified by 1,6-hexamethylene diisocyanate, toluene diisocyanate, 4,4′-diphenylmethane diisocyanate, and 4,4′-dicyclohexylmethane diisocyanate, respectively. A particularly preferred diisocyanate is hexamethylene diisocyanate (HMDI). Carbodiimide cross-linking agents may also be used, such as 1-ethyl-3-[3-dimethylaminopropyl]carbodiimide hydrochloride (EDC).

The extent to which the collagen-containing matrix is cross-linked may be varied. Usefully, this provides a mechanism for controlling the rate of resorption of the matrix following implantation. In general, the matrix should be sufficiently resistant to resorption to endure whilst host cells infiltrate the matrix and are subsequently influenced by the matrix to bring about guided tissue regeneration. It may be desirable that the collagen-containing matrix is resorbed to some extent over time, as part of the normal turnover of collagen and other fibrous matrix proteins at the site of implantation. The resistance to resorption tends to increase as the extent of cross-linking is increased.

By way of example, the matrix may be cross-linked using HMDI. As a guide, the HMDI may be used at a concentration of around 0.01 g to 0.5 g per 50 g of tissue. If the concentration is too high, this may result in over-cross-linking and foreign body reactions. It has been found that 0.1 g HMDI per 50 g of tissue provides good results. Cross-linking may be carried out for a range of different time periods. By way of example, the tissue may be exposed to the cross-linking agent for between around 1 hour and around 3 days. Typically, cross-linking is carried out for at least 12 hours, preferably at least 20 hours.

It will be appreciated that the cross-linking conditions may routinely be varied in order to adjust the extent of cross-linking.

In one preferred embodiment of the present invention, the tissue is treated with a solvent, preferably acetone, a proteolytic enzyme, preferably trypsin, and a cross-linking agent, preferably HMDI.

According to a further aspect of the present invention there is provided a method for guided tissue regeneration, said method including a step of implanting into a host a decellularised collagen-containing matrix as herein described.

According to a further aspect of the present invention there is provided the use of a decellularised collagen-containing matrix as herein described for guided tissue regeneration.

According to a further aspect of the present invention there is provided the use of a decellularised collagen-containing matrix as herein described in the manufacture of an implantable composition for guided tissue regeneration.

According to a still further aspect of the present invention there is provided the use of a process as herein described to produce a decellularised collagen-containing matrix for guided tissue regeneration.

Embodiments of the present invention will now be described further in the following non-limiting examples with reference to the accompanying drawings, in which:

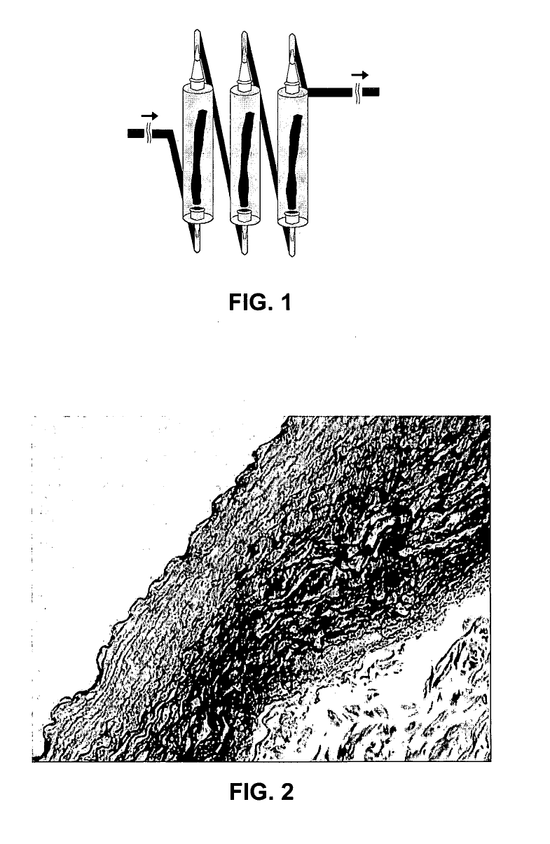

FIG. 1 is a diagrammatic representation of one type of tissue processing apparatus suitable for use in the present invention;

FIG. 2 is a photomicrograph (×200 magnification) of a section of a representative vascular matrix according to the present invention, stained with picrosirius red and Millers elastin stain.

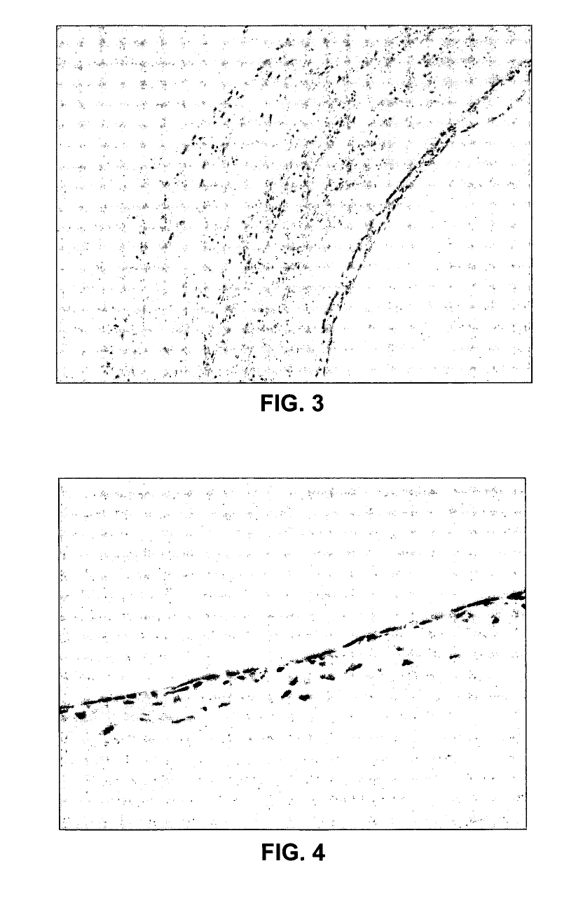

FIG. 3 is a photomicrograph (×200 magnification) of a section of a representative vascular matrix according to the present invention 7 days post-implantation in a porcine end-to-end carotid interpositional model, stained with haematoxylin and eosin;

FIG. 4 is a photomicrograph (×400 magnification) of a section of a representative vascular matrix according to the present invention 14 days post-implantation in a porcine end-to-end carotid interpositional model, stained with haematoxylin and eosin;

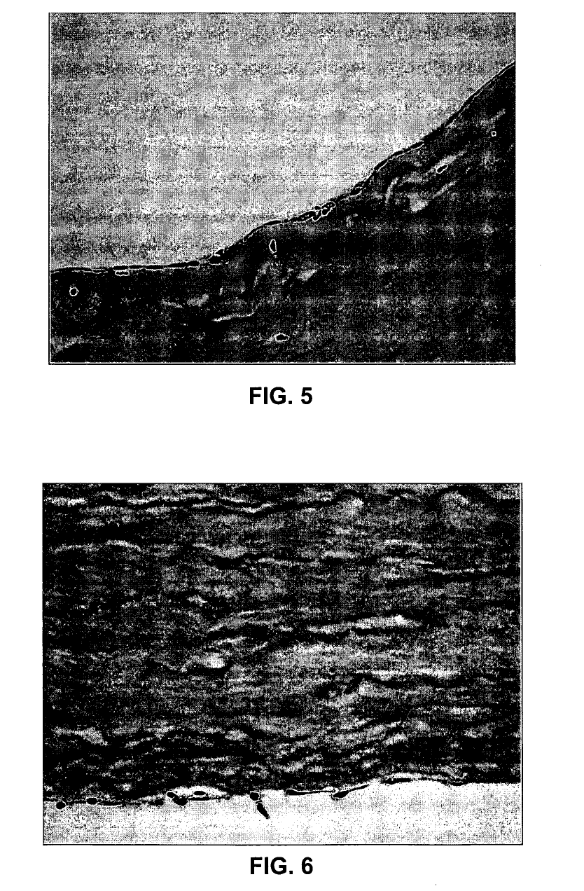

FIG. 5 is a photomicrograph (×400 magnification) of a section of a representative vascular matrix according to the present invention 28 days post-implantation in a porcine end-to-end carotid interpositional model, stained with haematoxylin and eosin;

FIG. 6 is a photomicrograph (×400 magnification) of a section of a representative vascular matrix according to the present invention 28 days post-implantation subdermally in a rat, stained with haematoxylin and eosin;

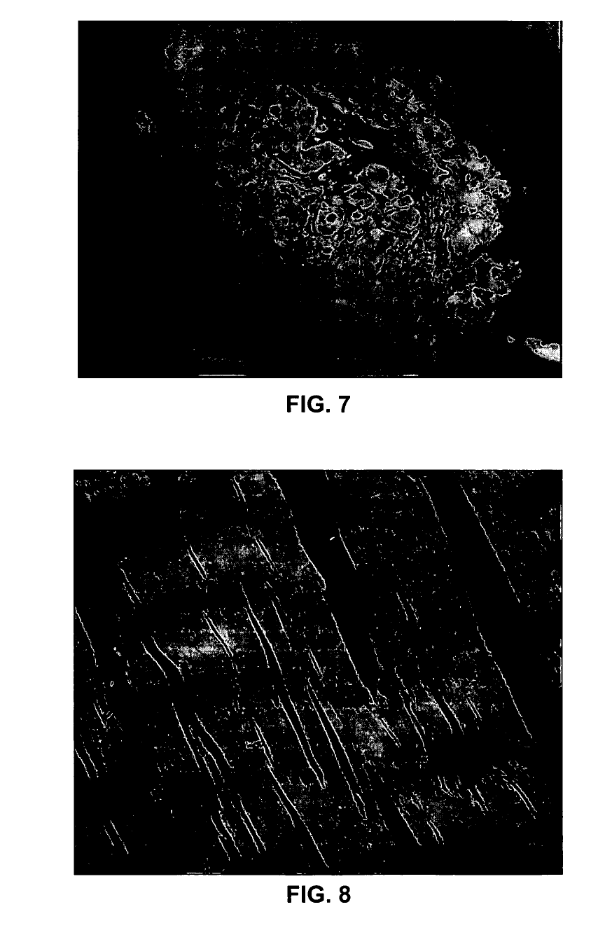

FIG. 7 is a photomicrograph (×400 magnification) of a section of a representative bone matrix according to the present invention 6 weeks post-implantation intramuscularly in a rat, stained with haematoxylin and eosin;

FIG. 8 is a polarised light micrograph (×200 magnification) of a longitudinal section of a representative tendon matrix according to the present invention, stained with picrosirius red and Millers elastin stain; and

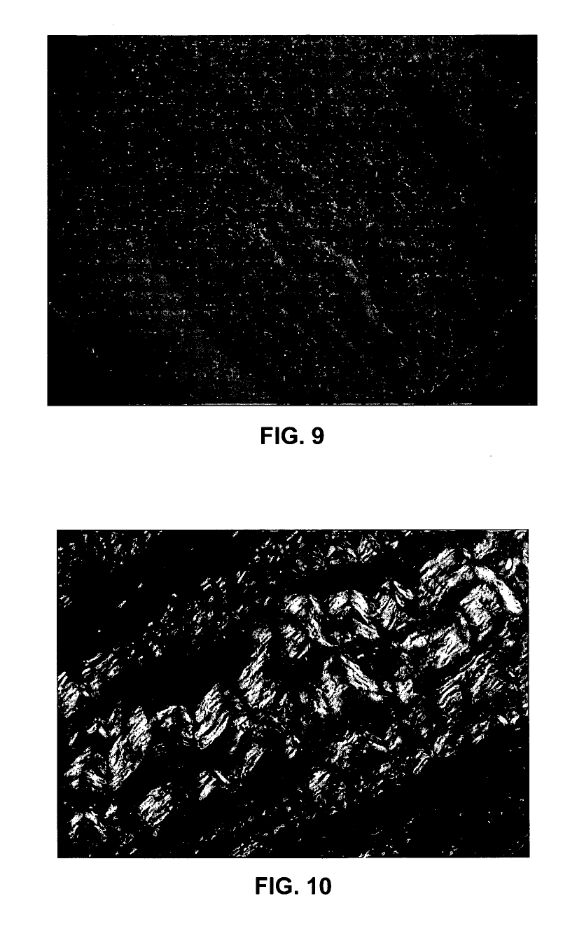

FIG. 9 is a photomicrograph (×200 magnification) of a section of a representative tendon matrix according to the present invention 6 weeks post-implantation subdermally in a rat, stained with haematoxylin and eosin.

FIG. 10 is a polarised light micrograph (×100) of a longitudinal section of a representative tendon matrix according to the present invention 6 weeks post implantation in a functional ovine anterior cruciate ligament model, stained with picrosirius red and Millers elastin stain.

### EXAMPLES

### 1. Matrix Prepared from Bone

Cancellous bone was harvested from the knee joint of a porcine hind limb. Harvesting was facilitated using a food grade band saw. All the cortical and cartilaginous material was cut from around the cancellous bone. The bone material was cut into pieces of around 1 cm³.

Upon completion of the harvesting process, the bone was then placed into acetone to remove lipids from the bone matrix. A 1-hour solvent rinse was followed by a 36-hour solvent rinse. The tissue was then rinsed thoroughly in 0.9% saline to remove the residual acetone from the structure. The material was then placed into trypsin at an activity of 2.5 g/L, for a total duration of 28 days, after which the material was washed with saline to rinse away residual trypsin. After completion of the trypsin digestion, the bone was rinsed thoroughly in saline. The material was then washed in acetone. There followed a cross-linking step of treatment with HDMI in acetone. The volume of HMDI required was based on an approximation of the quantity of collagen present in the bone tissue, calculated on a weight basis assuming that 30% of the bone tissue is collagen. A concentration of 0.1 g HMDI per 50 g of collagen was added. The material was cross-linked for at least 20 hours, rinsed in acetone, and finally rinsed in saline. Samples were then gamma-irradiated at 25 kGy.

For histological examination, samples were fixed in 10% neutral buffered formal saline. Following fixation, samples were processed, by routine automated procedures, to wax embedding. 10-micron resin sections were cut and stained with Giemsa. The sections of processed bone matrix showed the retention of cancellous structure, retention of calcium and were totally devoid of any cellular presence. All of the natural septae, the lacuna and the canaliculi showed no presence of any cellular or tissue material and were seen as empty clear spaces.

### 2. Intramuscular Implantation of Bone Matrix

Pieces of the decellularised collagen-containing bone matrix of Example 1 were implanted intramuscularly into rats. For implantation, slices of approximately 0.2 cm were cut from the 1 cm³ pieces of bone matrix.

Male Wistar rats were pre-medicated according to species and weight. General anaesthesia was induced and maintained using agents appropriate for species and size. Sterile technique was used. A dorsal cranio-caudal skin incision was made just lateral to the spine from a point 1 cm distal to the edge of the scapula extending approximately 1.5 cm distally. The psoas muscle was identified, exposed and divided longitudinally on each side to provide 2 intramuscular ‘pockets’. Haemostasis was maintained by careful dissection; no electrocautery was used. Samples of processed bone (approximately 1 cm×1 cm×0.2 cm) were implanted into each of the psoas muscle pockets. The psoas muscle pockets were closed with Vicryl® sutures and to complete the procedure the dorsal midline incision was then closed with interrupted sutures.

Six weeks after surgery, the implanted matrix was explanted together with the surrounding tissue and immediately fixed in 10% neutral buffered formal saline. Following fixation, samples were processed, by routine automated procedures, to wax embedding. 5-micron or 10-micron resin sections were cut and stained with Giemsa and/or haematoxylin and eosin.

The matrix was observed to be well integrated into the tissue, with no signs of an elevated immune response. There was a narrow band of mainly fibroblastic inflammatory response immediately adjacent to the matrix implant which occasionally extended a small distance into the muscle. Within this response there were some polymorphs, macrophages and the occasional monocyte. These features represent a normal ‘foreign body’ tissue response as would be seen with any non-immunogenic implant even an autograft. The implanted bone matrix retained its structure with easily definable morphological features, including calcified cancellous component and well preserved lacunae. The overall integrity of the matrix was also well preserved.

Within most of the lacunae, the septae and the cannaliculi of the implanted matrix samples there were thin, fibrinous, stranded structures within which there were a variety of cells including fibroblasts, polymorphs, monocytes and some larger mononuclear cells of indistinct lineage. In some of the lacunae there were large, mononuclear cells with recognisable nucleoli, which showed features of early osteocytic lineage (see FIG. 7). This was a surprising result, given that the tissue processing ostensibly renders the matrix inert, removing non-fibrous tissue proteins, such as growth factors. It would seem that the implanted bone matrix retained some signalling functionality. It was particularly surprising that this was apparently sufficient to influence the recruitment and/or development of osteocytic host cells in an intramuscular environment. Cells of this type would not be expected to be present at the host implant site. It is possible that the host cells were derived from progenitor cells, perhaps from the fibroblast milieu, although the exact mechanisms involved are unclear. The matrix may retain tissue-specific signals in elements of fibrous tissue protein sequence or conformation, which signals are able to influence host cell behaviour within the matrix, either directly or indirectly.

By way of further example an additional intramuscular study was completed comparing the bone matrix of Example 1 with Orthoss® and a demineralised version of the bone matrix of Example 1. Orthoss® is a commercially available bone implant derived from deproteinised bovine cancellous bone. Each of the materials for evaluation was trimmed to approximately 1 cm×1 cm×0.5 cm. These samples were separately implanted into intramuscular pockets on the latero-ventral aspect of rats. Samples were explanted at 2 months and at 3 months. Samples were explanted together with the adjacent surrounding tissues and fixed in 10% neutral buffered formal saline. Once fixed, the entire sample was de-calcified, a block from the centre of the explant, to include the implanted sample and all surrounding tissue, was processed to paraffin wax embedding by routine automated procedures. Two 5-micron sections were cut from each block, one was stained with haematoxylin and eosin and one with picrosirius red together with Millers elastin stain. Sections were examined using a transmitted light microscope with polarizing ability.

Both the demineralised bone matrix and Orthoss® elicited an immune reaction, with host cells breaking down the implanted devices.

The bone matrix of the present invention did not cause a foreign body inflammatory response and evidence of neo-collagenesis in the inter-trabecular spaces was identified. This may indicate early osteogenesis.

### 3. Matrix Prepared from Vascular Tissue

Carotid arteries (20-30 cm) were harvested from a porcine source. Upon completion of the harvesting process, the vessels were placed into acetone to remove lipids from the tissue. A 1-hour solvent rinse was followed by a 36-hour solvent rinse. The tissue was then rinsed thoroughly in 0.9% saline to remove the residual acetone from the structure. The material was then placed into trypsin at an activity of 2.5 g/L for 1 day, after which the material was washed with saline to rinse away residual trypsin. After completion of the trypsin digestion, the tissue was rinsed thoroughly in saline. The material was then washed in acetone. There followed a cross-linking step of treatment with HDMI in acetone. A concentration of around 0.1 g HMDI per 50 g of tissue was added. The material was cross-linked for at least 20 hours, rinsed in acetone, and finally rinsed in saline. Samples were then gamma-irradiated at 25 kGy.

Tissue processing was carried out in an apparatus as shown in FIG. 1, comprising a plurality of tubes connected in series. Processing solutions were pumped through the apparatus in the direction of the arrows.

A sample of the vascular matrix was fixed in 10% neutral buffered formal saline. Following fixation, the sample was processed, by routine automated procedures, to wax embedding. 5-micron resin sections were cut and stained using haematoxylin and eosin, picrosirius red and Millers elastin stain.

As shown in FIG. 2, the collagen and (darker-stained) elastin fibre structure is retained in the processed vascular matrix. The luminal surface of the vascular matrix is formed by the intact internal elastic lamella.

### 4. Subdermal Implantation of Vascular Matrix

Samples of vascular matrix prepared as in Example 3 were diametrically transected to produce implantable transverse pieces of matrix approximately 3 mm in length. Each sample consisted of a full transverse circle of matrix. Adult female Sprague Dawley rats were used at 250 g body weight as recipients for the collagen-containing matrix. In each animal, two subcutaneous pockets were formed lateral to the midline, one on each side, on the ventral aspect of the animal. For each of these subcutaneous pockets, a single transverse sample of vascular matrix was inserted, the pockets closed with a single Vicryl® suture and the midline incision closed with silk suture. At 7 and 28 days post-implantation, samples were explanted together with the surrounding tissue. Samples were fixed immediately in 10% neutral buffered formal saline. Following fixation, all samples were processed, by routine automated procedures, to wax embedding. Two 5-micron sections were cut from each sample; one was stained with haematoxylin and eosin and the other with a combination of picrosirius red and Millers elastin stain.

The collagen and elastin structure of the matrix was well preserved 7 days after subdermal implantation. The matrix demonstrated good biocompatibility after 7 days, with no significant chronic or acute inflammatory response and no other adverse cellular response. There was very good integration of the adventitial side of the vascular matrix with the local tissue.

It was also found that host endothelial cells were present on the internal lamella of the matrix when the samples were evaluated histologically after 7 days. The layer of endothelial cells was even better established after 28 days (see FIG. 6), with some evidence of cytoplasmic fusion. The endothelial cells tested positive for Von Willebrand factor.

The seeding of endothelial cells on the luminal surface of the collagen-containing matrix at the subdermal site was a surprising observation, in view of the lack of vasculature in the subdermal site of implantation or direct blood flow contact of the implanted matrix. The vascular matrix was treated to remove non-fibrous tissue proteins, such as growth factors, and was therefore considered to be essentially inert. However, it would seem that some signalling functionality was retained despite the tissue processing.

The reasons for this surprising result are not entirely clear. Again, it seems possible that the host cells may have responded to ‘signals’ provided by the structure of the collagen, elastin and/or other fibrous tissue proteins of the vascular matrix, resulting in recruitment and/or differentiation of host cells. The vascular matrix may retain tissue-specific signals in elements of fibrous tissue protein sequence or conformation, which signals are able to influence host cell behaviour within the matrix, either directly or indirectly, to give guided tissue regeneration.

### 5. Functional Implantation of Vascular Matrix

Samples of vascular matrix prepared as in Example 3 were used in an end-to-end carotid interpositional procedure in Large White/Landrace crossbred female pigs. The animals were pre-treated with an antithrombotic regime of 75 mg aspirin and 75 mg Clopidogrel. The animals were anaesthetised, intubated and ventilated throughout the procedure. Sterile technique was practised. A venous line was placed into a peripheral vein in the ear and glucose saline administered at 800 ml per hour throughout the procedure. A 15-20 cm midline access incision was made from chin to upper sternum. Right and left carotid arteries were exposed and isolated from surrounding tissue. Papaverine and 2% Procaine were administered topically to arteries to ensure vasodilation and 1000 units/kg of heparin were infused into a peripheral ear vein just prior to vessel clamping. The left carotid artery was clamped with single clamps followed by double clamping to provide a length of around 8-10 cm of exposed carotid artery between the clamps. Approximately 6 cm of this artery was resected using a vascular matrix of Example 3. The vascular matrix was interposed end-to-end into the natural artery and anastomosed with 6/0 or 8/0 continuous sutures. The distal clamps were removed and when the anastomoses stopped oozing the proximal clamps were removed. Pressure was applied until bleeding ceased. The procedure was repeated for the right side. Finally, the access incision was closed with two layers of 2/0 Vicryl® sutures internally and 2/0 Prolene® sutures externally. Ampicillin was administered at 25 mk/kg; Carprofen at 2-4 mg/kg with further doses for 2-3 days; and Ivomec at 0.02 ml/kg. The antithrombotic treatment was continued until harvesting.

After 7, 14 or 28 days, animals were anaesthetised as above and the grafts exposed by careful dissection. The vascular matrix was explanted together with the native proximal and distal carotid artery and immediately fixed in 10% neutral buffered formal saline. Following fixation, samples were processed, by routine automated procedures, to wax embedding. 5-micron resin sections were cut and stained using haematoxylin and eosin, picrosirius red and Millers elastin stain.

For comparison, the procedure was also carried out using venous autografts.

In the vein autografts, hyperplasia was observed after 7 days. By 14 days, hyperplasia was well advanced, and after 28 days following implantation hyperplasia was significant, the vessel becoming occluded as a result.

This is in contrast to the results observed using the vascular matrix according to the present invention. There was no significant chronic or acute inflammatory response and no other adverse cellular response was seen associated with any of the implanted samples.

The collagen and elastin structure of the vascular matrix was maintained 7 days after implantation in the end-to-end carotid interpositional procedure. At the 7-day stage, the external adventitial layer of the matrix had begun to integrate with the surrounding tissue, helping to stabilise the graft. There was no cell infiltration into the media of the matrix, and no smooth muscle proliferation or presence. Further, there was no evidence of thrombus formation and no platelet adherence to the luminal surface of the matrix. Even at this early stage, healthy endothelial cells had begun to seed onto the luminal surface of the graft (see FIG. 3), although not all of the luminal surface was populated with endothelial cells at the 7-day stage.

After 14 days, the collagen and elastin structure of the vascular matrix was maintained and the endothelial layer was better developed (see FIG. 4). Seeding of the endothelial layer was not from the ends of the graft, and so the cells would appear to be derived from circulating host endothelial cells and/or progenitor cells. Again, there was no evidence of smooth muscle cell proliferation. FIG. 4 shows that some of the endothelial cells had become characteristically cytoplasmically fused.

By 28 days, the collagen and elastin structure was still intact, including the internal elastic lamella. The endothelial layer was well established and present on almost all of the luminal surface of the graft (see FIG. 5). The endothelial cells appeared healthy and there was extensive cytoplasmic fusion. The adventitia was very well integrated into the host tissue and there were very few cells in the internal media of the matrix. There was some evidence of cell proliferation and/or remodelling beneath the endothelial layer. There may have been new tissue, perhaps basement membrane, laid down under the endothelium.

These results demonstrate that the collagen-containing matrix of the present invention functioned very well in practice, with no signs of thrombosis or intimal hyperplasia at up to four weeks post-implantation. The vascular matrix was readily seeded by host endothelial cells following implantation. It is suggested that the intact internal elastic lamella forming the luminal surface of the matrix may be important for achieving good endothelial regeneration. Further, the natural, ordered laying down of the new host endothelium following implantation seemingly results at least in part from the capacity of the matrix to induce guided tissue regeneration.

### 6. Matrix Prepared from Tendon

Flexor and extensor tendons were harvested from the hind limbs of porcine sows. Upon completion of the harvesting process, the tendons were dissected to remove extraneous connective tissue. They were then placed into acetone to remove lipids from the tendinous structure. A 1-hour solvent rinse was followed by a 36-hour solvent rinse. The tissue was then rinsed thoroughly in 0.9% saline to remove the residual acetone from the structure. The material was then placed into trypsin at an activity of 2.5 g/L for 3 days, after which the material was washed with saline to rinse away residual trypsin. After completion of the trypsin digestion, the tissue was rinsed thoroughly in saline. The material was then washed in acetone. There followed a cross-linking step of treatment with HDMI in acetone. A concentration of around 0.1 g HMDI per 50 g of tissue was added. The material was cross-linked for at least 20 hours, rinsed in acetone, and finally rinsed in saline. Samples were then gamma-irradiated at 25 kGy.

A sample of the tendon matrix was fixed in 10% neutral buffered formal saline. Following fixation, the sample was processed, by routine automated procedures, to wax embedding. 5-micron resin sections were cut and stained using haematoxylin and eosin.

The longitudinal fibre structure of the natural tendon tissue was retained in the processed matrix. Polarised light showed that the normal collagen banded structure was present in the matrix (FIG. 8).

### 7. Subdermal Implantation of Tendon Matrix

Samples of tendon matrix prepared as in Example 6 were implanted into adult female Sprague Dawley rats at 250 g body weight. In each animal, two subcutaneous pockets were formed lateral to the midline, one on each side, on the ventral aspect of the animal. For each of these subcutaneous pockets, a single piece of tendon matrix was inserted, the pockets closed with a single Vicryl® suture and the midline incision closed with silk suture. At 6 weeks post-implantation, samples were explanted together with the surrounding tissue. Samples were fixed immediately in 10% neutral buffered formal saline. Following fixation, all samples were processed, by routine automated procedures, to wax embedding. Sections of 5 microns were cut from the samples and stained using haematoxylin and eosin, picrosirius red and Millers elastin stain.

Histological examination showed infiltration of cells into the matrix. Cells with tenocyte-type morphology were observed, located in typical tendon-like patterns (FIG. 9). There was minimal inflammation, typical of a normal healing response. Again, these results are indicative of tissue regeneration guided by the tendon matrix.

### 8. Functional Implantation of Tendon Matrix

Tendon matrix prepared as in Example 6 was implanted for use in anterior cruciate ligament (ACL) reconstruction in an ovine model.

The Smith & Nephew Endobutton CL Fixation System for ACL reconstruction was used in conjunction with the tendon matrix and an Arthrex interferance screw. Two mature 2.5-3 year old ewes were used for the study. Before surgery, the force passing through both the animals' hind limbs was analysed by walking them over Kistler force plates. This assessed the load passing through the hind limbs and indicated whether, during gait, one leg was favoured over another. Anaesthesia was carried out using routine procedures and was maintained during the surgery by intubation and administration of halothane/O₂ mixture. Postoperatively, animals were given analgesics and antibiotics.

With leg in full extension a 10 cm incision starting at the right tibial tuberosity medial to the patellar tendon was made. The patella was disarticulated laterally. The fat pad was removed to expose the insertion of the ACL into the tibia. The insertion of the ACL into the femur was identified. With leg in flexion, a C guide (instrument specific for the sheep ACL model) was used to insert guide wire medially (about 1 cm) and below (about 1 cm) the tibial tuberosity, so that the guide wire emerged from the tibial plateau at the insertion point of the cruciate ligament. Cannulated drills were used over the wire to enlarge the tibial tunnel to 7-8 mm diameter. The rim of the tibial tunnel where it emerges into the joint was chamfered. Any remaining ACL inserting into the tibia was removed, i.e. the native ACL was completely removed.

The samples of tendon matrix of the invention were strap-like measuring 12-15 cm long, so that when assembled into a quad bundle the graft length measured approximately 3-4 cm. The matrix was trimmed as necessary so that the assembled quad bundle could pass through the bone tunnel (8 mm diameter).

With leg fully flexed, the femoral tunnel was prepared using a C guide and guide wire through femoral cruciate ligament insertion point so that it emerged on the lateral condyles.

The ligament graft was prepared by passing double bundle of the tendon matrix through the loop of the Endobutton and stitching the free ends together. The Endobutton was passed through the femoral tunnel and the tendon bundle tensioned. The stitched end of the tendon bundle was passed through the tibial tunnel. With the leg extended and the patella relocated, the bundle was tensioned and fixed in the tibial tunnel using a tunnel screw. Therefore reconstruction of the ACL was in the form of a graft consisting of a single quad-bundle and thus representative of current clinical practice for ACL reconstruction. The wound was closed and the animal allowed to recover and kept in a single pen.

Animals recovered so well that by 6 weeks post surgery there was no external evidence that their ACL had been replaced, i.e. there was no scarring or inflammation of the operative site. Furthermore the animals walked with normal gait.

Upon macroscopic evaluation of the explanted grafts it was clear there had been considerable remodelling of the tendon matrix with no evidence of separate bundles and it appeared as if a new ACL was forming.

The mid-section of the remodelled grafts were taken from both animals and processed for wax histology. The bone surrounding the insertion of the two grafts, adjacent to the femoral and tibial bone tunnels, was processed for decalcified histology.

Remnants of both grafts were visible at 6 weeks. The original fibres of the tendon matrix were evident, but appeared to be fragmented indicating that at this stage the graft was in the process of (adaptive) remodelling but that not all of the fibres had disappeared. The fibres were infiltrated with cells, some of which showed affinity with, and aligned to, the original porcine tendon matrix fibres, covering their entire surfaces. In these cases, cells appeared to behave as tenocyte-like cells.

In some regions where the original graft could not be seen, the well aligned fibrous tissue was associated with new crimped collagen fibres which could be clearly observed under polarised light (see FIG. 10). This form of collagen crimping is indicative of the natural ligament morphology. The remodelled graft in all regions where it was in the joint space was surrounded by a synovial-like layer of cells as in the natural ligament.

The presence of tenocyte-like cells and remodelling of the collagen matrix into a crimped ligamentous structure is surprising since the implanted matrix has no active factors present. Its remodelling and integration into a ligamentous tissue is another example of guided tissue regeneration.

It is of course to be understood that the invention is not intended to be restricted by the details of the above specific embodiments, which are provided by way of example only.

1. A decellularised collagen-containing matrix for guided tissue regeneration, wherein the matrix is derived from a natural tissue material and is substantially free of non-fibrous tissue proteins, cellular elements and lipids or lipid residues and wherein the matrix displays the original collagen fiber architecture and molecular ultrastructure of the natural tissue material from which it is derived.

2. A matrix according to claim 1, wherein the matrix comprises a portion of elastin.

3. A matrix according to claim 1, wherein the natural tissue material is a non-dermal tissue material.

4. A matrix according to claim 3, wherein the natural tissue material has more than one different collagen-containing components or sub-components.

5. A matrix according to claim 3, wherein the natural tissue material is selected from vascular tissue, bone, ligament, tendon, nerve, and bowel tissue.

6. A matrix according to claim 3, wherein the natural tissue material comprises an organ or a part thereof.

7. A matrix according to claim 6, wherein the organ is selected from heart, liver, kidney, pancreas, spleen, bladder, blood vessels, gastrointestinal tract, urethra, and ureter.

8. A matrix according to claim 1 for use as an implant for guided tissue regeneration.

9. An implant comprising a decellularised collagen-containing matrix, wherein the matrix is derived from a natural tissue material and is substantially free of non-fibrous tissue proteins, cellular elements and lipids or lipid residues and wherein the matrix displays the original collagen fiber architecture and molecular ultrastructure of the natural tissue material from which it is derived, characterised in that the matrix has a capacity to induce guided tissue regeneration.

10. A process for the manufacture of a decellularised collagen-containing matrix for guided tissue regeneration, which comprises treating a fibrous collagen-containing tissue material to remove therefrom cells and cellular elements, non-fibrous tissue proteins, lipids and lipid residues.

11. A process according to claim 10, wherein the fibrous collagen-containing tissue material comprises a portion of elastin.

12. A process according to claim 10, wherein the fibrous collagen-containing tissue material is a non-dermal tissue material.

13. A process according to claim 12, wherein the fibrous collagen-containing tissue material has more than one different collagen-containing components or sub-components.

14. A process according to claim 12, wherein the fibrous collagen-containing tissue material is selected from vascular tissue, bone, ligament, tendon, nerve, and bowel tissue.

15. A process according to claim 12, wherein the fibrous collagen-containing tissue material comprises an organ or a part thereof.

16. A process according to claim 15, wherein the organ is selected from heart, liver, kidney, pancreas, spleen, bladder, blood vessels, gastrointestinal tract, urethra, and ureter.

17. A process according to claim 10, wherein the process comprises a step of treatment with a proteolytic enzyme.

18. A process according to claim 17, wherein the proteolytic enzyme is trypsin.

19. A process according to claim 10, wherein the process comprises a step of removing lipids and lipid residues by solvent extraction using an organic solvent.

20. A process according to claim 19, wherein the solvent is selected from acetone, ethanol, ether, or mixtures thereof.

21. A process according to claim 10, wherein the process comprises a step of treatment with a cross-linking agent.

22. A decellularised collagen-containing matrix produced by a process according to claim 10.

23. A method for guided tissue regeneration, said method including a step of implanting into a host a decellularised collagen-containing matrix according to claim 1.

24. Use of a decellularised collagen-containing matrix according to claim 3 for guided tissue regeneration.

25. Use of a decellularised collagen-containing matrix produced by the process of claim 10 for guided tissue regeneration.

26. Use of a process according to claim 12 to produce a decellularised collagen-containing matrix for guided tissue regeneration.
